(19) United States
(12) Patent Application Publication
(10) Pub. No.: US 2025/0191693 A1
(43) Pub. Date: Jun. 12, 2025

(54) MACHINE-LEARNING TECHNIQUES IN PROTEIN DESIGN FOR VACCINE GENERATION

(71) Applicant: Sanofi Pasteur, Inc., Swiftwater, PA (US)

(72) Inventors: Philip Davidson, Cambridge, MA (US); Maryann Giel-Moloney, Cambridge, MA (US); Konstantin Zeldovich, Cambridge, MA (US)

(21) Appl. No.: 18/845,375

(22) PCT Filed: Mar. 10, 2023
(86) PCT No.: PCT/US2023/014965

§ 371 Date: Sep. 9, 2024

Related U.S. Application Data
(60) Provisional application No. 63/319,700, filed on Mar. 14, 2022.
(60) Provisional application No. 63/319,692, filed on Mar. 14, 2022.

(51) Int. Cl. G16B 40/00

(52) U.S. Cl. CPC G16B 40/00

(57) ABSTRACT

One or more data objects are received defining a plurality of wild-type amino acid sequences. From the one or more data objects, a plurality of reduced-dimension sequences are generated in a reduced-dimension space. A plurality of candidate sequences are generated in the reduced-dimension space using the plurality of reduced-dimension sequences. One or more data objects defining a viral amino acid sequence are received. Viral sequences in the reduced-dimension space are received. As input to a titer-predictor, each of the candidate sequences and at least one of the reduced-dimension viral sequences are provided. As output from the titer-predictor, a candidate-score for each of the candidate sequences is received. At least one candidate sequence from among the candidate sequences are selected. At least one new amino acid sequence is generated. Each of the generated amino acid sequences is suitable for manufacturing a respective vaccine.

### CROSS REFERENCE TO RELATED APPLICATIONS

This application claims priority to U.S. Provisional Application No. 63/319,700, filed on Mar. 14, 2022 and U.S. Provisional Application No. 63/319,692, filed on Mar. 14, 2022, the entire contents of which are incorporated herein by reference.

### TECHNICAL FIELD

This application is related to use of machine learning techniques in the design of vaccines.

### BACKGROUND

Machine learning (ML) is the use of computer algorithms that can improve automatically through experience and by the use of data. It is seen as a part of artificial intelligence. Machine learning algorithms build a model based on sample data, known as training data, in order to make predictions or decisions without being explicitly programmed to do so. Machine learning algorithms are used in a wide variety of applications, such as in medicine, email filtering, speech recognition, and computer vision, where it is difficult or unfeasible to develop conventional algorithms to perform the needed tasks.

A vaccine is a biological preparation that provides acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a natural or “wild” pathogen), or therapeutic (to fight a disease that has already occurred, such as cancer). Some vaccines offer full sterilizing immunity, in which infection is prevented completely.

### SUMMARY

The strains used in seasonal influenza vaccines are currently and near-universally chosen by public health authorities. These selections are made yearly, based on observations of immune response in animal models and human studies. However, H3N2 vaccines using the strains recommended by the public health authorities have not been sufficient to elicit broad protection in the general population, e.g., over the past 5 years. Further, during this time-frame public data shows that immunological relatedness has split into divergent clades wherein each clade is protective to itself while protection against other clades can be limited. The present disclosure provides a solution to this problem. The implementations described in this disclosure provide for an algorithm that can generate influenza (or other) antigens for use as a vaccine. In one implementation, this can include:
1) Generating a reduced-dimension space for all wildtype hemagglutinin sequences through machine learning (e.g., variational autoencoder architecture) using two steps:
   a. Embedding variably into a reduced space, e.g. model predicts mean and variance from input sequence, embedded coordinates selected from normal distribution with predicted mean and variance.
   b. Decoding back to original sequence from reduced space location “autoencoder” loss function is then performed, reducing by the similarity of the input and output sequences.
2) Training an immune response prediction model based on location of antigen (vaccine candidate) and readout strains (target sequences) in the reduced dimensional space [input: antigen and readout embedded by model of step 1, output: measure of immune response such as antibody titer].
3) Sampling candidate vaccine component representations from the reduced space, rank candidate vaccine component representations by predicted performance against target sequences using the model described in step 2, and identify top candidates.
4) Decoding top candidate representations [using model from step 1b] to emit hemagglutinin sequences that may or may not have been observed in the original wildtype set.

In an example experiment, the algorithm was used to optimize the HAI sequence of H3 hemagglutinin (positions 16 to 345) and then wildtype signal peptide and HA2 regions were grafted on to create a complete hemagglutinin sequence. An exemplary modified antigen sequence starting from A/Singapore/INFIMH-16-0019/2016 is provided with mutated residues indicated in bold:

(SEQ ID NO: 1)
MKTIIALSYILCLVFAQKIPGNDNSTATLCLGHHAVPNGTIVKTITNDR
IEVTNATELVQNSSIGEICDSPHQILDGENCTLIDALLGDPQCDGFQNK
KWDLFVERSKAYSNCYPYDVPDYASLRSLVASSGTLEFNNESFNWTGVT
QNGTSSACIRKSSSSFFSILNWLTHLNYTYPALNVTMPNKEQFDKLYIW
GVHHPGTDKDQISLYARSSGRITVSTKRSQQAVIPNIGSRPRIRDIPSR
ISIYWTIVKPGDILLINSTGNLIAPRGYFKIRSGKSSIMRSDEPIGKCK
SECITPNGSIPNDKPFQNVNRITYGACPRYVKHSTLKLATGMRNVPEKQ
TRGIFGAIAGFIENGWEGMVDGWYGFRHQNSEGRGQAADLKSTQAAIDQ
INGKLNRLIGKTNEKFHQIEKEFSEVEGRIQDLEKYVEDTKIDLWSYNA
ELLVALENQHTIDLTDSEMNKLFEKTKKQLRENAEDMGNGCFKIYHKCD
NACIGSIRNGTYDHNVYRDEALNNRFQIKGVELKSGYKDWILWISFAIS
CFLLCVALLGFIMWACQKGNIRCNICI

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a dimension-reducing method for generating amino acid sequences, the method being performed by a system of one or more computers. The method includes receiving one or more data objects defining a plurality of wild-type amino acid sequences. The method also includes generating, from the one or more data objects, a plurality of reduced-dimension sequences in a reduced-dimension space, where: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space. The method also includes generating a plurality of candidate sequences in the reduced-dimension space using the plurality of reduced-dimension sequences. The method also includes receiving one or more data objects defining a viral amino acid sequence. The method also includes generating at least one reduced-dimension viral sequences in the reduced-dimension space. The method also includes providing, as input to a titer-predictor, each of the candidate sequences and at least one of the reduced-dimension viral sequences. The method also includes receiving, as output from the titer-predictor, a candidate-score for each of the candidate sequences. The method also includes selecting at least one candidate sequence from among the candidate sequences. The method also includes generating at least one new amino acid sequence for each of the selected candidate sequences. The method also includes providing the generated at least one amino acid sequence. The method also includes operations where each of the generated amino acid sequences is suitable for manufacturing a respective vaccine may include at least one of the group that may include of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The method includes operations where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. The method includes operations where n is a value of 1, such that a single candidate sequence is selected. The method includes operations where n is a value greater than 1, such that a plurality of candidate sequences are selected. Selecting the at least one candidate sequence as a selected candidate sequence may include selecting candidate sequences with respective candidate-scores greater than a threshold value. Each of the generated amino acid sequences is different from any of the wild-type amino acid sequences. At least one of the candidate sequences is in the plurality of reduced-dimension sequences. The respective vaccine is for one of the group that may include of i) influenza, ii) human rhinovirus, iii) hiv and iiiv) a coronavirus disease. Implementations of the described techniques may include hardware, a method or process, or computer software on a computer-accessible medium.

One general aspect includes a system for generating amino acid sequences, the system may include computer memory. The system also includes one or more processors. The system also includes computer-memory storing instructions that, when executed by the processors, cause the processors to perform operations that may include: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of the group consisting of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The system where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. Implementations of the described techniques may include hardware, a method or process, or computer software on a computer-accessible medium.

One general aspect includes a non-transitory, computer readable media storing instructions that, when executed by one or more processors, cause the one or more processors to perform operations including: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of the group consisting of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The media where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Implementations of the described techniques may include hardware, a method or process, or computer software on a computer-accessible medium.

Also disclosed herein are vaccine compositions comprising a plurality of any of the generated amino acid sequences of the methods described herein.

Also disclosed are vectors, fusion proteins, and cells comprising one or more of the peptides and/or proteins produced according to the methods described herein.

Also disclosed herein are methods of eliciting an immune response in a subject that include administering one or more of the isolated nucleic acids, peptides and/or proteins described herein, thereby eliciting an immune response in the subject.

In one aspect, disclosed herein are methods of inhibiting a viral infection that includes administering to a subject any of the one or more isolated nucleic acids, peptides and/or proteins described herein or any of the vaccines comprising any of the isolated nucleic acids, peptides and/or proteins described herein.

Also disclosed herein are methods of immunizing a subject against influenza virus comprising administering to the subject an immunologically effective amount of the vaccine composition as disclosed herein. Also disclosed herein is a vaccine composition as disclosed herein for use in a method of immunizing a subject against a virus (e.g., an influenza virus). Also disclosed herein is a vaccine composition as disclosed herein for the manufacture of a medicament for use in a method of immunizing a subject against a virus (e.g., an influenza virus). In certain embodiments, the method prevents a viral infection (e.g., an influenza virus infection) in a subject, and in certain embodiments, the method raises a protective immune response (e.g., an HA antibody response and/or an NA antibody response), in the subject. In certain embodiments, the subject is human, and in certain embodiments, the vaccine composition is administered intramuscularly, intradermally, subcutaneously, intravenously, or intraperitoneally.

Another aspect of the disclosure is directed to a method of reducing one or more symptoms of a viral infection (e.g., an influenza virus infection), the method comprising administering to a subject a prophylactically effective amount of the vaccine composition disclosed herein. Also disclosed herein is a vaccine composition as disclosed herein for use in a method of reducing one or more symptoms of a viral infection (e.g., an influenza virus infection). Also disclosed herein is a vaccine composition as disclosed herein for the manufacture of a medicament for use in a method of reducing one or more symptoms of an infection (e.g., an influenza virus infection).

In various embodiments, the methods and compositions disclosed herein treat or prevent disease caused by either or both a seasonal or a pandemic viral strain (e.g., a seasonal or pandemic influenza strain).

In certain embodiments of the methods disclosed herein wherein the subject is human, the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age. For example, the subject is 6 months, 8 months, 10 months, 12 months, 14 months, 16 months, 18 months, 20 months, 22 months, 24 months, 3 years, 4 years, 5 years, 6 years, 10 years, 12 years, 15 years 18 years, 20 years, 21 years, 25 years, 30 years, 35 years, 40 years, 50 years, 60 years, 70 years, 75 years, 80 years, 85 years, or 90 years old. In certain embodiments, the methods disclosed herein comprise administering to the subject two doses of the vaccine composition with an interval of 2-6 weeks, such as an interval of 4 weeks.

The implementations discussed in this disclosure can provide one or more of the following advantages. The implementations can be used to generate hemagglutinin sequences with potential to induce broad protection from influenza infection following vaccination. Notably, the implementations can be used to produce antigens that have a greater than expected recovery rate of functional influenza virus with designed hemagglutinin sequences. These antigens are believed to have broad protection, greater than current standard of care antigens in an animal model. The implementations can be used to generate broadly protective hemagglutinin proteins for use as influenza vaccine antigens.

By converting amino acid sequences into lower dimensional space and adding variation into their representations, new and non-wildtype amino acid sequences can be generated. These new amino acid sequences can then be used to manufacture new and more efficacious vaccines than would otherwise be possible. For example, there may exist non-wildtype amino acid sequences that cause a subject (e.g., a human) to produce a more potent immune response when exposed to a vaccine with proteins of the non-wildtype amino acid sequences, leading to greater protection and greater health.

Another advantage of the techniques provided in the present disclosure is to improve likelihood of generating protein sequence data for proteins that can actually exist and be manufactured. As will be understood, it is possible to describe protein sequences that, due to geometry, physical forces, etc., cannot exit. Processes described in this document can be advantageously constrained to only those known to or expected to be manufacturable.

Other features, aspects and potential advantages will be apparent from the accompanying description and figures.

### DESCRIPTION OF DRAWINGS

FIG. 1 is a block diagram of an example system that can be used to manufacture a vaccine.

FIG. 2 is a schematic diagram of data that can be used in the manufacture of a vaccine.

FIGS. 3-6 are flowcharts of example processes that can be used to process high-dimensional data in lower dimensional space, such as may be used in the manufacture of a vaccine.

FIG. 7 is a swimlane diagram of an example process to manufacture a vaccine.

FIG. 8 is a schematic diagram that shows an example of a computing device and a mobile computing device.

Like reference symbols in the various drawings indicate like elements.

### DETAILED DESCRIPTION

This document describes vaccine creation through machine learning processes. The vaccine creation uses candidate proteins that are generated by a computational process that includes machine learning. A corpus of wildtype amino acid sequences are provided to a variable autoencoder to produce a low-dimensional representation (latent space) of sequences. After training of such a model, some representations, when decoded, may generate non-wildtype amino acid sequences. Representations in the latent space are tested to identify one or more representations that are computationally predicted to generate an amino acid sequence that will produce a desired biological response in a subject. One or more of these candidate representations are selected based on the predicted desired response, and are converted to the higher-dimensional space of traditional amino acid sequence definitions. A vaccine or vaccines are manufactured from these newly defined amino acid sequence or sequences. Sequences may be filtered to exclude non-wildtype or wildtype sequences, as needed.

Influenza virus is a member of the Orthomyxoviridae family. There are three subtypes of influenza viruses: influenza A, influenza B, and influenza C. Influenza A viruses infect a wide variety of birds and mammals, including humans, chickens, ferrets, pigs, and horses. In mammals, most influenza A viruses cause mild localized infections of the respiratory and intestinal tract.

The influenza virion contains a negative-sense RNA genome, which encodes the following nine proteins: hemagglutinin (HA), matrix (M1), proton ion-channel protein (M2), neuraminidase (NA), nonstructural protein 2 (NS2), nucleoprotein (NP), polymerase acidic protein (PA), polymerase basic protein 1 (PB1), and polymerase basic protein 2 (PB2). The HA, M1, M2, and NA are membrane associated proteins, whereas NP, NS2, PA, PB1, and PB2 are nucleocapsid associated proteins. The M1 protein is the most abundant protein in influenza particles. The HA and NA proteins are envelope glycoproteins, which are responsible for virus attachment and cellular entry. The HA and NA proteins are the source of the major immunodominant epitopes for virus neutralization and protective immunity. The HA and NA proteins are considered the most important components for prophylactic influenza vaccines.

HA is a viral surface glycoprotein that generally comprises approximately 560 amino acids and representing 25% of the total virus protein.

NA is a membrane glycoprotein of the influenza viruses. NA is 413 amino acid in length, and is encoded by a gene of 1413 nucleotides. Nine different NA subtypes have been identified in influenza viruses (N1, N2, N3, N4, N5, N6, N7, N8 and N9), all of which have been found among wild birds.

The influenza virus' ability to cause widespread disease stems from its ability to evade the immune system by undergoing antigenic change.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Adjuvant: As used herein, the term “adjuvant” refers to a substance or combination of substances that may be used to enhance an immune response to an antigen component of a vaccine.

Antigen: As used herein, the term “antigen” refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, ferrets, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the NA and HA forms as described herein.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Epitope: As used herein, the term “epitope” includes any moiety that is specifically recognized by an immunoglobulin (e.g., antibody or receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, sorbitol, gelatin, malt, rice, flour, chalk, silica gel, sodium stearate, glycerol monostearate, talc, sodium chloride, dried skim milk, glycerol, propylene, glycol, water, ethanol and the like.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (prevents infection or prevents the development of disease associated with infection) or reduces the symptoms of infection. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to render a subject protected from an infectious disease.

Immunologically effective amount: As used herein, the term “immunologically effective amount” means an amount sufficient to immunize a subject.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Vaccination: As used herein, the term “vaccination” or “vaccinate” refers to the administration of a composition to generate an immune response, for example to a disease-causing agent such as an influenza virus. Vaccination can be administered before, during, and/or after exposure to a disease-causing agent, and/or to the development of one or more symptoms, and in some embodiments, before, during, and/or shortly after exposure to the agent. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response. In some embodiments, vaccination includes multiple administrations, appropriately spaced in time, of a vaccinating composition.

Vaccine Efficacy: As used herein, the term “vaccine efficacy” or “vaccine effectiveness” refers to a measurement in terms of percentage of reduction in evidence of disease among subjects who have been administered a vaccine composition. For example, a vaccine efficacy of 50% indicates a 50% decrease in the number of disease cases among a group of vaccinated subjects as compared to a group of unvaccinated subjects or a group of subjects administered a different vaccine.

Wild type (WT): As is understood in the art, the term “wild type” generally refers to a normal form of a protein or nucleic acid, as is found in nature. For example, wild type HA and NA polypeptides are found in natural isolates of influenza virus. A variety of different wild type HA and NA sequences can be found in the NCBI influenza virus sequence database.

### Measuring Hemagglutinin Activity

Hemagglutinin activity may be measured using techniques known in the art, including, for example, hemagglutinin inhibition assay (HAI). An HAI applies the process of hemagglutination, in which sialic acid receptors on the surface of red blood cells (RBCs) bind to a hemagglutinin glycoprotein found on the surface of an influenza virus (and several other viruses) and create a network, or lattice structure, of interconnected RBCs and virus particles, referred to as hemagglutination, which occurs in a concentration dependent manner on the virus particles. This is a physical measurement taken as a proxy as to the facility of a virus to bind to similar sialic acid receptors on pathogen-targeted cells in the body. The introduction of anti-viral antibodies raised in a human or animal immune response to another virus (which may be genetically similar or different to the virus used to bind to the RBCs in the assay) interfere with the virus-RBC interaction and change the concentration of virus sufficient to alter the concentration at which hemagglutination is observed in the assay. One goal of an HAI can be to characterize the concentration of antibodies in the antiserum or other samples containing antibodies relative to their ability to elicit hemagglutination in the assay. The highest dilution of antibody that prevents hemagglutination is called the HAI titer (i.e., the measured response).

Another approach to measuring a HA antibody response is to measure a potentially larger set of antibodies elicited by a human or animal immune response, which are not necessarily capable of affecting hemagglutination in the HAI assay. A common approach for this leverages enzyme-linked immunosorbent assay (ELISA) techniques, in which a viral antigen (e.g., hemagglutinin) is immobilized to a solid surface, and then antibodies from the antisera are allowed to bind to the antigen. The readout measures the catalysis of a substrate of an exogenous enzyme complexed to either the antibodies from the antisera, or to other antibodies which themselves bind to the antibodies of the antisera. Catalysis of the substrate gives rise to easily detectable products. There are many variations of this sort of in vitro assay. One such variation is called antibody forensics (AF), which is a multiplexed bead array technique that allowed a single sample of serum to be measured against many antigens simultaneously. These measurements characterize the concentration and total antibody recognition, as compared to HAI titers, which are taken to be more specifically related to interference with sialic acid binding by hemagglutinin molecules. Therefore, an antisera's antibodies may in some cases have proportionally higher or lower measurements than the corresponding HAI titer for one virus's hemagglutinin molecules relative to another virus's hemagglutinin molecules; in other words, these two measurements, AF and HA1, may not be linearly related.

Another method of measuring HA antibody response includes a viral neutralization assay (e.g., microneutralization assay), wherein an antibody titer is measured by a reduction in plaques, foci, and/or fluorescent signal, depending on the specific neutralization assay technique, in permissive cultured cells following incubation of virus with serial dilutions of an antibody/serum sample.

### Measuring Neuraminidase Activity

Neuraminidase activity can be measured using techniques known in the art, including, for example, a MUNANA assay, ELLA assay, or an NA-Star® assay (ThermoFisher Scientific, Waltham, MA). In the MUNANA assay, 2′-(4-methylumbelliferyl)-alpha-D-N-acetylneuraminic acid (MUNANA) is used as a substrate. Any enzymatically active neuraminidase contained in the sample cleaves the MUNANA substrate, releasing 4-Methylumbelliferone (4-MU), a fluorescent compound. Thus, the amount of neuraminidase activity in a test sample correlates with the amount of 4-MU released, which can be measured using the fluorescence intensity (RFU, Relative Fluorescence Unit).

For purposes of determining the neuraminidase activity of a soluble tetrameric NA of the present disclosure, a MUNANA assay should be performed using the following conditions: mix soluble tetrameric NA with buffer [33.3 mM 2-(N-morpholino) ethanesulfonic acid (MES, pH 6.5), 4 mM CaCl2, 50 mM BSA] and substrate (100 uM MUNANA) and incubate for 1 hour at 37° C. with shaking; stop the reaction by adding an alkaline pH solution (0.2M Na2CO3); measure fluorescence intensity, using excitation and emission wavelengths of 355 and 460 nm, respectively; and calculate enzymatic activity against a 4MU reference. If necessary, an equivalent assay can be used to measure neuraminidase enzymatic activity.

### Vaccine Compositions

In certain aspects, disclosed herein is a vaccine composition comprising a plurality of generated amino acid sequences.

Each generated amino acid sequence may be present in the compositions disclosed herein in an amount effective to induce an immune response in a subject to which the composition is administered. In certain embodiments, each generated amino acid sequence may be present in the vaccine compositions disclosed herein in an amount ranging, for example, from about 0.1 g to about 500 g, such as from about 5 g to about 120 g, from about 1 g to about 60 g, from about 10 g to about 60 g, from about 15 g to about 60 g, from about 40 g to about 50 g, from about 42 g to about 47 g, from about 5 g to about 45 g, from about 15 g to about 45 g, from about 0.1 g to about 90 g, from about 5 g to about 90 g, from about 10 g to about 90 g, or from about 15 g to about 90 g. In certain embodiments, each recombinant HA may be present in the vaccine compositions disclosed herein in an amount of about 5 g, 10 g, 15 g, 20 g, 25 g, 30 g, 35 g, 40 g, 45 g, 50 g, 55 g, 60 g, 65 g, 70 g, 75 g, 80 g, 85 g, or about 90 g.

The vaccine composition can also further comprise an adjuvant. As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO4), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, AS04, an Aluminum Salt- and TLR4 Agonist-Based Adjuvant System, Induces a Transient Localized Innate Immune Response Leading to Enhanced Adaptive Immunity, J. IMMUNOL. 2009, 183:6186-6197), IL-2, RANTES, GM-CSF, TNF-?, IFN-?, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein 90% of the population by volume of the oil drops has a size less than 200 nm.

In certain embodiments, the polyoxyethylene alkyl ether is of formula CH3—(CH2)x—(O—CH2—CH2)n—OH, in which n is an integer from 10 to 60, and x is an integer from 11 to 17. In certain embodiments, the polyoxyethylene alkyl ether surfactant is polyoxyethylene(12) cetostearyl ether.

In certain embodiments, 90% of the population by volume of the oil drops has a size less than 160 nm. In certain embodiments, 90% of the population by volume of the oil drops has a size less than 150 nm. In certain embodiments, 50% of the population by volume of the oil drops has a size less than 100 nm. In certain embodiments, 50% of the population by volume of the oil drops has a size less than 90 nm.

In certain embodiments, the adjuvant further comprises at least one alditol, including, but not limited to, glycerol, erythritol, xylitol, sorbitol and mannitol.

In certain embodiments the hydrophilic/lipophilic balance (HLB) of the hydrophilic nonionic surfactant is greater than or equal to 10. In certain embodiments, the HLB of the hydrophobic nonionic surfactant is less than 9. In certain embodiments, the HLB of the hydrophilic nonionic surfactant is greater than or equal to 10 and the HLB of the hydrophobic nonionic surfactant is less than 9.

In certain embodiments, the hydrophobic nonionic surfactant is a sorbitan ester, such as sorbitan monooleate, or a mannide ester surfactant. In certain embodiments, the amount of squalene is between 5 and 45%. In certain embodiments, the amount of polyoxyethylene alkyl ether surfactant is between 0.9 and 9%. In certain embodiments, the amount of hydrophobic nonionic surfactant is between 0.7 and 7%. In certain embodiments, the adjuvant comprises: i) 32.5% of squalene, ii) 6.18% of polyoxyethylene (12) cetostearyl ether, iii) 4.82% of sorbitan monooleate, and iv) 6% of mannitol.

In certain embodiments, the adjuvant further comprises an alkylpolyglycoside and/or a cryoprotective agent, such as a sugar, in particular dodecylmaltoside and/or sucrose.

In certain embodiments, the adjuvant comprises AF03, as described in Klucker et al., AF03, an alternative squalene emulsion-based vaccine adjuvant prepared by a phase inversion temperature method, J. PHARM. SCI. 2012, 101 (12): 4490-4500, which is hereby incorporated by reference in its entirety. In certain embodiments, the adjuvant comprises a liposome-based adjuvant, such as SPA14. SPA14 is a liposome-based adjuvant (AS01-like) containing a toll-like receptor 4 (TLR4) agonist (E6020) and saponin (QS21).

In addition to the recombinant HAs, recombinant NAs, and optional adjuvant, the vaccine composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine composition is a sterile, liquid solution formulated for parenteral administration, such as intravenous, subcutaneous, intraperitoneal, intradermal, or intramuscular. The vaccine composition may also be formulated for intranasal or inhalation administration. The vaccine composition can also be formulated for any other intended route of administration.

In some embodiments, a vaccine composition is formulated for intradermal injection, intranasal administration or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19th ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the vaccine composition is administered using a device that delivers a metered dosage of the vaccine composition. Suitable devices for use in delivering intradermal pharmaceutical compositions described herein include short needle devices such as those described in U.S. Pat. No. 4,886,499, U.S.

U.S. Pat. Nos. 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO 1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, U.S. U.S. Pat. No. 4,940,460, WO 1997/37705, and WO 1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Kits

Further disclosed herein are kits for the vaccine compositions as disclosed herein. Kits may include a suitable container comprising the vaccine composition or a plurality of containers comprising different components of the vaccine composition, optionally with instructions for use.

In certain embodiments, the kit may comprise a plurality of containers, including, for example, a first container comprising one or more isolated nucleic acids, peptides and/or proteins as disclosed herein.

### Nucleic Acids, Cloning, and Expression Systems

The present disclosure further provides artificial nucleic acid molecules. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, or a derivative thereof, such as pseudouridine, unless context requires otherwise. Other nucleotide derivatives or modified nucleotides can be incorporated into the artificial nucleic acid molecules.

The present disclosure also provides constructs in the form of a vector (e.g., plasmids, phagemids, cosmids, transcription or expression cassettes, artificial chromosomes, etc.) comprising an artificial nucleic acid molecule encoding the generated amino acid sequences as disclosed herein. The disclosure further provides a host cell which comprises one or more constructs as above.

Also provided are methods of making the isolated peptides and/or proteins using recombinant techniques known in the art and as discussed above. The production and expression of recombinant proteins is well known in the art and can be carried out using conventional procedures, such as those disclosed in Sambrook et al., Molecular Cloning: A Laboratory Manual (4th Ed. 2012), Cold Spring Harbor Press. For example, expression of the HA or NA polypeptide may be achieved by culturing under appropriate conditions host cells containing the artificial nucleic acid molecule encoding the HA or

NA as disclosed herein. For example, expression of the recombinant HA or NA polypeptide may be achieved by culturing under appropriate conditions host cells containing the nucleic acid molecule encoding the HA or NA as disclosed herein. Following production by expression, the HA or NA may be isolated and/or purified using any suitable technique, then used as appropriate.

Systems for cloning and expression of a polypeptide in a variety of different host cells are well known in the art. Any protein expression system (e.g., stable or transient) compatible with the constructs disclosed in this application may be used to produce the generated amino acid sequences described herein.

Suitable vectors can be chosen or constructed, so that they contain appropriate regulatory sequences, including promoter sequences, terminator sequences, polyadenylation sequences, enhancer sequences, marker genes and other sequences as appropriate.

For expressing the generated amino acid sequences as disclosed herein, nucleic acids encoding the generated amino acid sequences can be introduced into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. These techniques are well known in the art. (See, e.g., “Current Protocols in Molecular Biology,” Ausubel et al. eds., John Wiley & Sons, 2010). DNA introduction may be followed by a selection method (e.g., antibiotic resistance) to select cells that contain the vector.

The host cell may be a plant cell, a yeast cell, or an animal cell. Animal cells encompass invertebrate (e.g., insect cells), non-mammalian vertebrate (e.g., avian, reptile and amphibian) and mammalian cells. In one embodiment, the host cell is a mammalian cell. Examples of mammalian cells include, but are not limited to COS-7 cells, HEK293 cells; baby hamster kidney (BHK) cells; Chinese hamster ovary (CHO) cells; mouse sertoli cells; African green monkey kidney cells (VERO-76); human cervical carcinoma cells (e.g., HeLa); canine kidney cells (e.g., MDCK), and the like. In one embodiment, the host cells are CHO cells. In one embodiment, the host cells are insect cells.

### Methods of Use

The present disclosure provides methods of administering the vaccine compositions described herein to a subject. The methods may be used to vaccinate a subject against a virus (e.g., an influenza virus). In some embodiments, the vaccination method comprises administering to a subject in need thereof a vaccine composition comprising one or more isolated nucleic acids, peptides and/or proteins encoding the generated amino acid sequences as described herein (e.g., recombinant influenza virus Has as described herein or recombinant influenza virus NAs as described herein), and an optional adjuvant in an amount effective to vaccinate the subject against a virus (e.g., an influenza virus). Likewise, the present disclosure provides a vaccine composition comprising one or more isolated nucleic acids, peptides and/or proteins encoding the generated amino acid sequences described herein (e.g., influenza virus Has or NAs as as described herein), and an optional adjuvant, for use in (or for the manufacture of a medicament for use in) vaccinating a subject against a virus (e.g., an influenza virus).

The present disclosure also provides methods of immunizing a subject against a virus (e.g., an influenza virus), comprising administering to the subject an immunologically effective amount of a vaccine composition comprising one or more recombinant influenza virus HAs or NAs as described herein, and an optional adjuvant.

In some embodiments, the method or use prevents a viral infection (e.g., an influenza virus infection) or disease in the subject. In some embodiments, the method or use raises a protective immune response in the subject. In some embodiments, the protective immune response is an antibody response.

The methods/use of immunizing provided herein can elicit a broadly neutralizing immune response against one or more viruses (e.g., influenza viruses). Accordingly, in various embodiments, the composition described herein can offer broad cross-protection against different types of viruses (e.g., influenza viruses). In some embodiments, the composition offers cross-protection against avian, swine, seasonal, and/or pandemic influenza viruses. In some embodiments, the methods/use of immunizing are capable of eliciting an improved immune response against one or more seasonal influenza strains (e.g., a standard of care strain). For example, the improved immune response may be an improved humoral immune response. In some embodiments, the methods/use of immunizing are capable of eliciting an improved immune response against one or more pandemic influenza strains. In some embodiments, the methods of immunizing are capable of eliciting an improved immune response against one or more swine influenza strains. In some embodiments, the methods/use of immunizing are capable of eliciting an improved immune response against one or more avian influenza strains.

In certain embodiments, provided herein are methods of enhancing or broadening a protective immune response in a subject, the method comprising administering to the subject an immunologically effective amount of the vaccine composition disclosed herein, wherein the vaccine composition increases the vaccine efficacy of a standard of care influenza virus vaccine composition by an amount ranging from about 5% to about 100%, such as from about 10% to about 25%, from about 20% to about 100%, from about 15% to about 75%, from about 15% to about 50%, from about 20% to about 75%, from about 20% to about 50%, or from about 40% to about 80%, such as about 40% to about 60% or about 60% to about 80%. In certain embodiments, the vaccine composition disclosed herein has a vaccine efficacy that is at least 5% greater than the vaccine efficacy of a standard of care influenza virus vaccine, such as a vaccine efficacy that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the vaccine efficacy of a standard of care influenza virus vaccine. Likewise, the present disclosure provides any of the vaccine compositions described herein for use in (or for the manufacture of a medicament for use in) enhancing or broadening a protective immune response in a subject.

Also provided are methods of preventing a viral disease (e.g., an influenza virus disease) in a subject, comprising administering to the subject a vaccine composition comprising one or more isolated nucleic acids, peptides and/or proteins encoding the generated amino acid sequences (e.g., recombinant influenza virus HAs or NAs as described herein), and an optional adjuvant in an amount effective to prevent a viral disease (e.g., an influenza virus disease) in the subject. Likewise, the present disclosure provides a vaccine composition comprising one or more recombinant influenza virus HAs or NAs as described herein, and an optional adjuvant, for use in (or for the manufacture of a medicament for use in) preventing a viral disease (e.g., an influenza virus disease) in a subject.

Also provided are methods of inducing an immune response against an influenza virus HA and an influenza virus NA in a subject, comprising administering to the subject a vaccine composition comprising one or more recombinant influenza virus HAs as described herein, one or more recombinant influenza virus NAs as described herein, and an optional adjuvant.

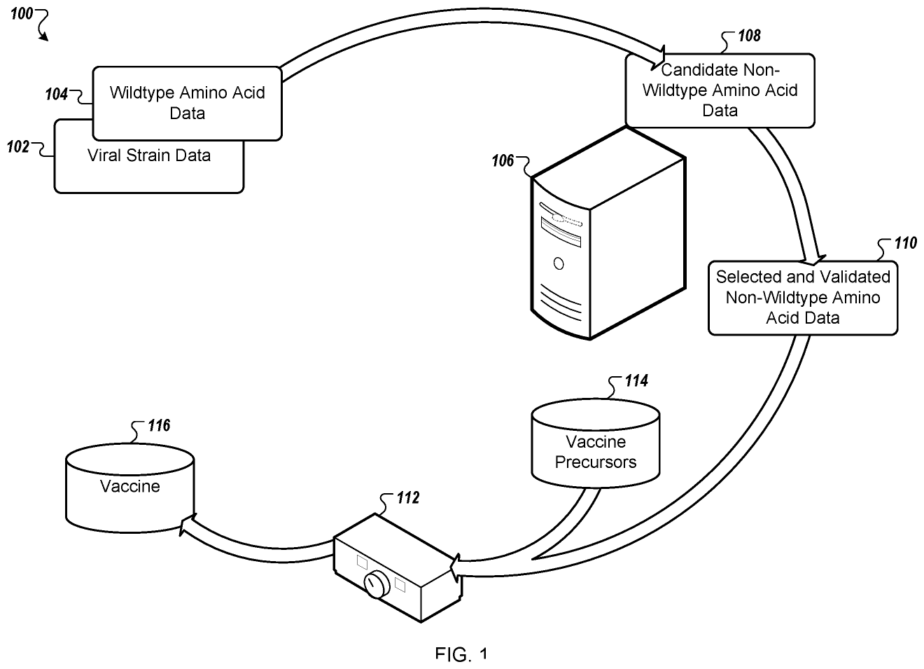

FIG. 1 is a block diagram of an example system 100 that can be used to manufacture a vaccine. In the system 100, a new vaccine 116 is designed and manufactured using technology described in this document. For example, for a virus with many strains and/or strains that mutate quickly such as influenza, human rhinovirus, HIV, a coronavirus virus such as coronavirus disease 2019, or for new viruses never before encountered, the technology described here can be used to quickly generate vaccine candidates that can be tested for use in humans or other subjects.

As input, system 100 receives viral strain data 102, and wildtype amino acid data 104. Viral strain data 102 includes data about one or more viral strains for which vaccines are desired. This viral strain data 102 can include amino acid sequence data, as well as other types of data such as metadata (e.g., unique identifiers, strain identification) or non-metadata properties (e.g., records of physiochemical properties of the amino acid sequence such as molecular weight). The wildtype amino acid data 104 can include a corpus of amino acid definitions for hundreds, thousands, hundreds of thousands, or more amino acid sequences. These sequences are referred to here as wildtype, indicating that in some embodiments they are generally amino acid sequences found in the wild. However, in other embodiments, amino acid sequences may include artificial amino acid sequences never before seen, sequences of human-manufactured amino acids, or other types of amino acids. The amino acid data 104 can include amino acid sequence data, as well as other types of data such as metadata (e.g., unique identifiers, strain identification) or non-metadata properties (e.g., records of physiochemical properties of the amino acid sequence such as molecular weight).

System 100 includes computer system 106 that can generate data 108 of candidate non-wildtype amino acid sequences by using the data 102 and 104. These non-wildtype amino-acid sequences are amino acids that are not found in the wild, or that are not known to be found in the wild. As will be appreciated, it is possible that one or more candidate non-wildtype amino acid sequences 108 may be in-fact in the wild, but not known to the operators of the system 100 or even to the community at large. The candidate non-wildtype amino acid data 108 can include amino acid sequence data, as well as other types of data such as metadata (e.g., unique identifiers, strain identification) or non-metadata properties (e.g., records of physiochemical properties of the amino acid sequence such as molecular weight).

Computer system 106 validates one or more of the candidates in the data 108 for manufacture, resulting in data 110. The data 110 can include amino acid sequence data, as well as other types of data such as metadata (e.g., unique identifiers, strain identification) or non-metadata properties (e.g., records of physiochemical properties of the amino acid sequence such as molecular weight). In some cases, the data 102/104, 108, and 110 are in the same data format, and in some cases the data 102/104, 108, and 110 are in different data formats.

In some cases, the validation process used to select candidates can include determining if the amino acid sequence can be synthesized at all, or if it can be synthesized easily or economically. As will be appreciated, it is possible for an amino acid sequence to define a structure of a molecule that cannot exist in the physical world due to the geometry and forces such a molecule would exhibit. As such, such impossible sequences can be excluded from the validation process. In addition, some of the candidates may be excluded even though they define valid molecules. For example, the computing system 106 can maintain a datastore of previous candidates that failed to actually be effective as a vaccine once investigated in clinical trials or predicted to be less immunogenic or less protective against viral strains of interest, which may include viral strain data 102. In such a case, candidates in the data 108 can be excluded from the validated data 110. In some cases, candidates can be excluded or prioritized based on synthetization and manufacturing considerations. For example, a candidate with particular synthesizing or handling conditions (e.g., cold storage, shock sensitivity) can be excluded from validation or deprioritized compared to other candidates having less onerous synthesizing or handling conditions.

System 100 can also include vaccine manufacturing devices 112 that can use vaccine precursors 114 and one or more validated non-wildtype amino acid sequence data 110 to manufacture one or more vaccine doses or vaccine molecules 116. As will be understood, initial exploration and testing would call for much smaller-scale synthesizing than large-scale manufacturing of a vaccine that has been tested, found safe and effective, and approved for use in humans or other subjects. Therefore, the particulars of the manufacturing devices 112 can vary according to the needs. Similarly, while the vaccine precursors 114 include those articles, chemicals, materials, etc. for the manufacture of the vaccine 116, the precursors 114 will similarly vary according to the needs.

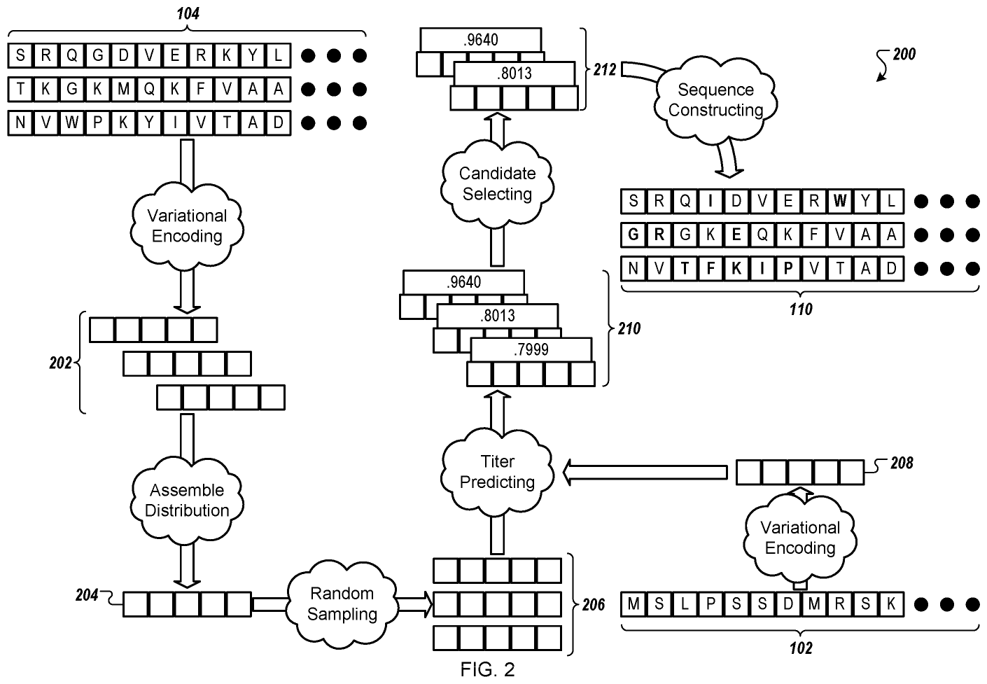

FIG. 2 is a schematic diagram of data that can be used in the manufacture of a vaccine. For example, the data shown here can be used by the computer system 106 or other computer systems. In broad strokes, the data 104 is transformed into a lower-dimension space, modified to make new amino acid sequences, and then one or more of those are selected for use in vaccine manufacture. This data may be used by the computer system 106 or other computing systems.

Wildtype amino acid data 104 is one or more data objects defining a plurality of wild-type amino acid sequences. Wildtype amino acid data 104 is shown here with a subsection of some of the sequences rendered for legibility using the single-letter designation recommended by the International Union of Pure and Applied Chemistry-International Union of Biochemistry and Molecular Biology (IUPAC-IUBMB) Joint Commission on Biochemical Nomenclature. For wildtype amino acid sequences, the data 104 can include a vector of data values (e.g., single American Standard Code for Information Interchange (ASCII) characters, an integer) to represent the amino acids in the sequence represented by the data 104. As will be appreciated, longer sequences will have more indices than those shown visually here, and more sequences may be stored in the data 104 that what is shown. In addition, other portions of the data 104 are not rendered here for clarity. Each amino acid sequence can be recorded as either single letters or letter strings. The letter strings can include multiple single letters. The one or more amino acid sequences can include a first amino acid sequence and a second amino acid sequence, each of the first and the second amino acid sequences including respective single letters or respective letter strings. That is to say, each amino acid sequence can be stored in data that conforms to the same format, while holding different values. This can allow for interoperability and consistent handling of the data.

As will be appreciated, the vectors of the data 104 have a length, which may all be the same across vectors. These vectors define a certain dimensionality of the data 104. For example a length of 632 defines a space with 632 dimensions, a length of 88 defines a space with 88 dimensions, etc. For sequences that can contain one of 20 different amino acids, each dimension has a domain or size of 20. Therefore, the corpus of amino acid sequences in the data 104 define a distribution of vectors (or point locations) in a space with dimensionality of the length of the amino acid sequence.

The data 104 undergoes variational encoding (described elsewhere) and produces, from one or more data objects, a plurality of reduced-dimension sequences 202 in a reduced-dimension space. In this example, the reduced-dimension space has 5 dimensions, and the data 202 can be recorded in vectors of length 5, however different dimensionality (and thus length of vectors) can be used in other implementations. The data 202 can record data (e.g., real numbers) or other appropriate data in each index of the vectors, with the values encoded from the values in the amino acid sequences of the data 104 with variational data added from the variational encoding. In some implementations, these real numbers are trained such that 1) similar sequences will be proximal, 2) numeric coordinates can be decoded using the decoder portion of the model. Therefore, each reduced-dimension sequence contains data respective of at least one of the wild-type amino acid sequence

In some examples, this variational encoding includes a lossy data transformation, resulting in data 202 that is based on, but does not contain all of the information in, the data 104. However, this is nevertheless an advantageous process because it can allow for the operations described in this document that produce new, non-wildtype amino acid sequences useful for vaccine development and manufacture.

The reduced-dimension space is of a lower dimensionality than the corresponding wild-type amino acid sequences. This can allow for computational operations in the lower dimensionality space that would be impossible, computationally inefficient, or otherwise undesirable in the higher dimension space of the data 104. For example, because the dimensions of the reduced-dimension space are not collinear to a dimension in the higher dimension space, or do not represent a small subset of the higher dimension space, properties of a single dimension do not map directly onto a single dimension of the higher dimensional space. Thus, operations in a single dimension of the reduced-dimension space allow for efficient execution and can produce results that are impossible or not intuitive when operating or thinking in the higher dimension space.

In some implementations, data 206 stores candidate sequences generated from the data 202. One such example random sampling is performed from the entire normally distributed space defined by the data 202. For example, if there are 5 dimensions in the data 202, there are 5 axes to select normally across.

In some implementations, data 206 stores candidate sequences generated from interstitial data 204 One such example is the assembling of distributions in each of the dimensions of the reduced-dimension space. Because the data 202 is stored as a plurality of vectors, statistical distributions of the values in a particular dimension can be assembled in data 204. For example, if integers are stored in each index of the vectors in data 202, each index of the vector of data 204 can store a histogram of the integers in the same index in the vectors in data 202. In another example, if real numbers are stored in each index of the vectors in data 202, each index of the vector of data 204 can store parameters of a function defining a best fit curve such as would be found via linear regression or similar analysis. As will be appreciated, the type of data stored in each index of the data 204 can be determined based on the type of data stored in each index of the data 202. In this way the plurality of reduced-dimension sequences define a distribution of values along each dimension of the reduced-dimension space.

Data 206 stores candidate sequences. For example, a plurality of candidate-sequences can be generated in the same reduced-dimension space that is used for the data 202 and 204. This can be performed many times (tens, hundreds, thousands, tens of thousands, hundreds of thousands, millions, billions, trillions, or more) to create many candidate sequences. Data 206 can store definitions of amino acid sequences that have similar properties to those in the data 104 (though stored in the lower-dimension space like the data 202), but that may not actually show up in the data 104. If the sequences in the data 104 have particularly beneficial or desirable properties, those properties can be expected to be found in at least some of the data 206. For example, if the amino acid sequences in the data 104 elicit an immune response in a subject, sequences defined by the data 206 are likely to provide a similar immune response. And, since they are different than the amino acids in the sequence 104 to a certain extent, they are likely to elicit a greater or lesser immune response. As such, as will be explained, this can produce new sequences, never before known or never before appreciated, that elicit a greater immune response, making them better for use in vaccines. In such a way, the technology of vaccines is beneficially advanced.

In some cases, the data 204 is randomly sampled according to its distributions to create the data 206. For example, if each index contains a histogram, a value in the histogram is selected weighted by the height of each value in the histogram. For example, if each index contains parameters for a curve defining a Y value given an X value, an X value can be selected weighted by the height of the curve or by randomly selecting one of the points under the curve. In such a way, the distribution values in a given index in the data 202 will be similar to the distribution of values in the same index in the data 206, though it is statistically unlikely they will be identical.

Each of the candidate sequences in the data 206, as will be described, can then be tested and the best candidates selected for analysis or use in vaccine manufacture. For example, an immune response predictor such as antibody titer predictor can be used to predict the immune response in a subject for a given viral amino acid sequence. The titer predictor may be configured to accept, as input, two amino acid sequences. This function may be configured to return, as output, a predicted immune response of a subject (e.g., human, animal). This output may take the form of, for example, a value between 0 and 1, with higher values indicating a prediction of greater immunity response. This predictor function may operate using a machine-learning model.

To perform this operation, the data 102 contain the viral amino acid sequence is modified in the same way that the data 104 is, resulting in forming data 208 that is in the same format and of the same kind as the data 202. That is to say, the data 102 holding one or more data objects defining a viral amino acid sequence undergoes variational encoding (described elsewhere) and produces a reduced-dimension viral sequence 208. This data 208 stores data in the same reduced-dimension space as the data 202-206, allowing for efficient computational operations on any of the data 202-208.

For each sequence in the data 206, the titer predictor produces a candidate-score. The candidate score is the predicted immune response for the amino acid sequence. Shown here are three examples of the many sequences and scores, though it will be appreciated that the titer predictor can used many time (tens, hundreds, thousands, tens of thousands, hundreds of thousands, millions, billions, trillions, or more) to create as many candidate-scores as there are candidate sequences.

These candidate-scores indicate a predicted level of immune response, and therefore can be thought of as a prediction of how effective the candidate-sequence is likely to be in a vaccine. At least one selected candidate sequence is selected from the candidate-sequences. Various computational process may be used to identify the ‘best’ sequences for testing and/or manufacture.

In one example, a pre-defined or dynamically-defined number of candidate sequences are selected. This involves selection of the candidate sequences with N highest candidate-scores. The value of N may be based, for example, on the throughput of apparatuses and systems capable of testing vaccines, so if N amino acid sequences can be tested, the same N value can be used here. In cases where Nis a value greater than 1, a plurality of candidate sequences are selected. In cases where Nis a value of 1, a single candidate sequence is selected. Shown here is an example in which N has a value of 2 such that 2 sequences are selected.

In one example, a pre-defined or dynamically-defined threshold value is used to select candidate sequences. This threshold value may be based, for example, on a minimum value expected to be able to produce good results. As will be appreciated, this threshold value may be near the maximum value (e.g., near but less than 1) to select only the most promising candidate sequences, near the minimum value (e.g., near but greater than 0) to select all but the least promising candidates, or any other appropriate value. This may result, in some cases, of selection no candidate sequences, depending on the threshold and the quality of the candidates.

Data 110 is created by constructing amino acid sequences in the higher dimension space used by the data 102 and 104. Depending on the configurations of the operations, it may be possible that a single representation in the data 212 may map to two or more amino acid sequences. As previously described, transforming from the high-dimension space to the low-dimension space may be lossy. In some such cases, this may mean that any given sequence representation in the low-dimension space may be ambiguous and specify two or more actual amino acids. In the example shown, one candidate sequence is used to create one new amino acid sequence while another candidate sequence is used to create two new amino acid sequences, though more than two may also be possible.

Due to the constraints of the data processing described, the new amino acid sequences in data 110 can retain a certain amount of similarity (e.g., as defined by an edit distance or other metric) to the wildtype amino acid sequences in the data 104. For clarity, differences between the data 104 and 110 have been rendered by making certain letters in the data 110 bold.

It will be appreciated that it is possible, but not required, that one of the new amino acid sequences in the data 110 are the same as one of the wildtype amino acid sequences in the data 104. Furthermore, it is possible, but not required, that one of the new amino acid sequences 110 is the same as a wildtype amino acid sequence found in nature and not involved in the data operations described in this document. Further, it is possible, but not required, that one of the new amino acid sequences 110 is the same as another new amino acid sequence that was previously created with this or another data processing operation, tested for potential as a vaccine, and discarded (e.g., due to low efficacy, safety concerns, an inability to manufacture, or other undesirable property). Therefore, in some cases, the data 110 may be filtered to remove new amino acid sequences that are known, leaving only unknown or unanalyzed amino acid sequences.

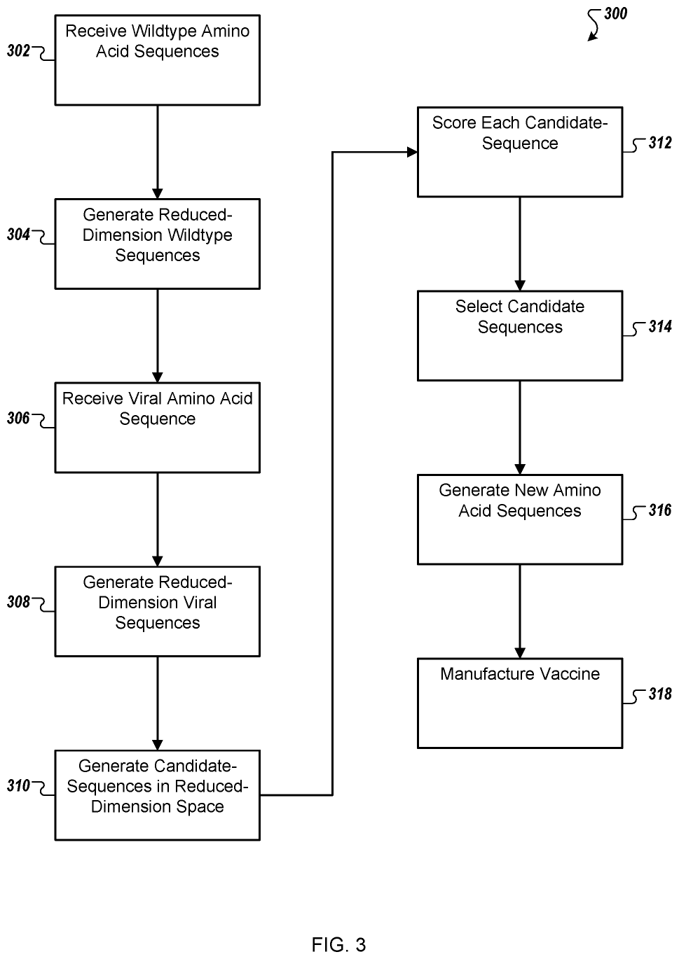

FIG. 3 is a flowchart of an example process 300 that can be used to process high-dimensional data in lower dimensional space, such as may be used in the manufacture of a vaccine. For example, the process 300 can be performed using the data shown in FIGS. 1 and 2, e.g., 102/104, 110, 202-212, and will therefore use elements of those figures in the description. Possible embodiments of various elements of the process 300 are described later in processes 400-700.

One or more data objects defining a plurality of wildtype amino acid sequences are received 302. For example, the computer system 106 accesses the data 104 from a disk, receives the data 104 over a network connect, etc.

I A plurality of reduced-dimension sequences are generated in a reduced-dimension space 304. For example, the computer system 106 can use one or more data-processing operation that uses, as input, the data 104 and produces, as output, the data 202. In doing so, the computer system 106 can embed variability into the data 202. An example of 304 will be described in greater detail in process 400.

One or more data objects defining a viral amino acid sequence are received 306. For example, the computer system 106 accesses the data 102 from a disk, receives the data 104 over a network connection, etc.

At least one reduced-dimension viral sequence in the reduced-dimension space is generated 308. For example, the computer system 106 can use one or more data-processing operation that uses, as input, the data 102 and produces, as output, the data 208. In some instances, the computer system 106 can embed variability into the data 208 in the same way as is performed in 304 (see, e.g., process 400). In other examples, the computing system embeds variation differently or not at all.

A plurality of candidate-sequences in the reduced-dimension space are generated using the plurality of reduced-dimension sequences 310. For example, the computer system 106 can analyses the data 202 to generate the data 204. To do so, the computer system 106 can characterize the values of the various vectors of the data 202 and record such characterizations in the data 204. In some cases, the computer system 106 creates plurality of candidate-sequences in the reduced-dimension space by sampling distributions of values of the plurality of reduced-dimension sequences.

Each candidate-sequence is scored to produce candidate-scores 312. For example, the computer system 106 can analyze the data 206 and 208 to generate the data 210. In some cases, the computer system 106 may use a predictor or classifier that has been trained on historic data of amino acids in the low-dimension space to generate predictions of biological response (e.g., how strongly a subject produces antibodies). An example of 312 will be described in greater detail in process 500.

At least one candidate sequence is selected as a selected candidate sequence 314. For example, the computing system 106 can generate the data 212 from the data 210. The computing system 106 can use, for example, the candidate-scores to select the selected candidate sequences. An example of 314 will be described in greater detail in process 600.

At least one new amino acid sequence for each selected candidate sequence is generated 316. For example, the computing system 106 can generate the data 110 from the data 212 and provide the data 110 to be used for manufacture of a vaccine. To do so, the computing system 106 can find points or vectors in the high-dimension space that correspond to the points or vectors in the data 212. As will be appreciated, projecting vectors from a low dimensional space to a high dimensional space may define an area of result instead of a single point-result. Therefore the computer system 106 may in some cases generate every valid amino acid sequence that is in the area of result, leading to more than one new amino acid sequence in data 110 for each vector in data 212.

A vaccine for each new amino acid sequence is manufactured 318. The vaccine can comprise a protein defined by the new amino acid sequence and/or a nucleic acid, or any other delivery vehicle including viral or bacterial vectors, whereby such nucleic acid or delivery vehicle produces the protein defined by the new amino acid sequence 318. For example, the computing device 106 and/or the vaccine manufacturing devices 112 can operate to use the data 110 and vaccine precursors 114 to create the vaccine 116. This manufacture may be a small batch for purposes of initial test, for clinical trials, and/or for general use. As will be appreciated, the elements 316 and 318 may be separated by a significant amount of time and interstitial operations. For example, if the manufacturing in 318 is large-volume manufacture for general use, this may be only after clinical trials have demonstrated that the vaccine is safe and effective for its intended purpose.

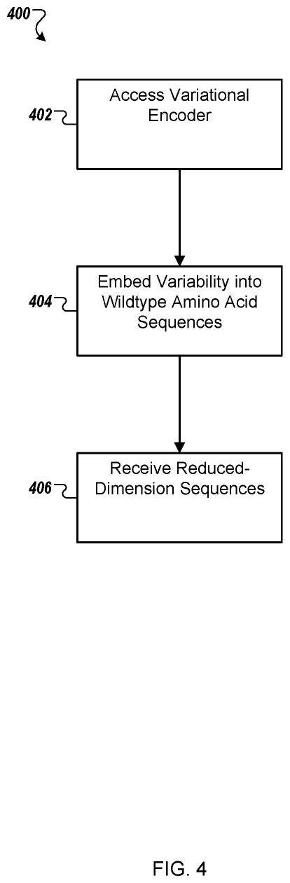

FIG. 4 is a flowchart of an example process 400 that can be used to process high-dimensional data in lower dimensional space, such as may be used in the manufacture of a vaccine including the creation of representations of the wild-type amino acid sequences using a variational autoencoder that predicts mean and variance values of input data. For example, the process 400 can be performed using the data shown in FIGS. 1 and 2 and will therefore use elements of those figures in the description. The process 400 is a possible example of how operation 304 may be performed, though other processes may be used.

One or more variational autoencoders (which are discussed in further details below) are accessed 402. For example, the computer system 106 accesses the data for the autoencoders from a disk, receive the data over a network connect, etc. This data can define one or more functions, libraries, modules, etc. that operate on input data and return output data.

The variational autoencoder 404 creates a low-dimensional representation of amino acid sequences. For example, the computing system 106 can execute the variational autoencoder with the data 104 to create the data 202.

Reduced-dimension sequences are received 406. For example, the computing system 106 can receive the data 202 from the variational autoencoder, which can include accessing the data from a disk, receives the data 202 over a network connect, etc.

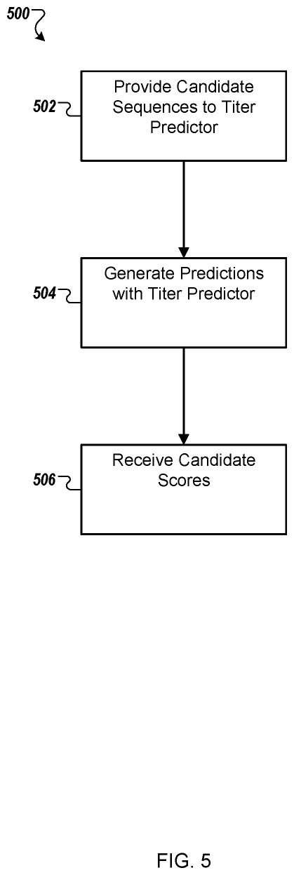

FIG. 5 is a flowchart of an example process 500 that can be used to process high-dimensional data in lower dimensional space, such as may be used in the manufacture of a vaccine. For example, the process 500 can be performed using the data shown in FIGS. 1 and 2 and will therefore use elements of those figures in the description. The process 500 is a possible example of how operation 312 may be performed, though other processes may be used.

Each of the candidate sequences and the reduced-dimension viral sequence are provided as input to an antibody titer-predictor 502. For example, the computer system 106 accesses the data for the titer predictor from a disk, receive the data over a network connect, etc. This data can define one or more functions, libraries, modules, etc. that operate on input data and return output data. This operation can be performed sequentially for one or more reduced-dimension viral sequences.

Predictions are generated with the titer-predictor 504. For example, the computing system 106 can execute the titer predictor with the data 206 and 208 to create the data 210.

A candidate-score for each candidate-sequence is received as output from the titer-predictor 506. For example, the computing system 106 can receive the data 210 from the titer predictor, which can include accessing the data from a disk, receives the data 202 over a network connect, etc.

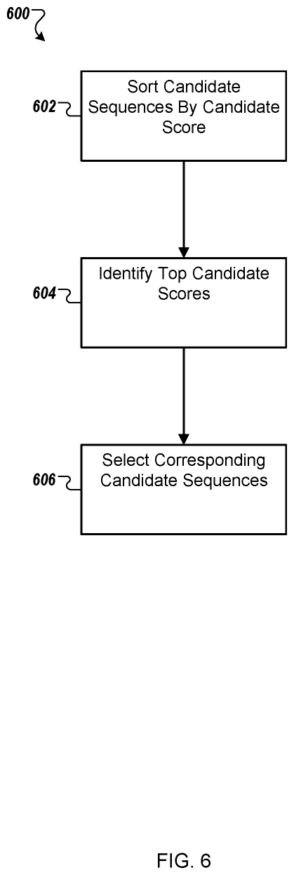

FIG. 6 is a flowchart of an example process 600 that can be used to process high-dimensional data in lower dimensional space, such as may be used in the manufacture of a vaccine. For example, the process 600 can be performed using the data shown in FIGS. 1 and 2 and will therefore use elements of those figures in the description. The process 600 is a possible example of how operation 314 may be performed, though other processes may be used.

Candidate-sequences are sorted by candidate-score 602. For example, the computer system 106 can order, in memory the data 210 into a list so that each entry in the list has a greater than or equal (or lesser than or equal) candidate-score than the entry after it.

Top candidate scores are identified 604. For example, the computer system 106 can identify some of the pairs of candidate sequence: candidate score from the beginning (or end) of the list. In some cases, the computer system 106 selects N pairs with the highest candidate-scores, where Nis some positive integer value. In some cases, the computer 106 selects all pairs with a candidate-score greater than a threshold value, where the threshold value is less than the greatest possible candidate-score and greater than the minimum possible candidate-score.

Candidate sequences corresponding to the top candidate scores are selected 606. For example, the computer system 106 can select the candidate sequence in the sequence: candidate score pairs identified.

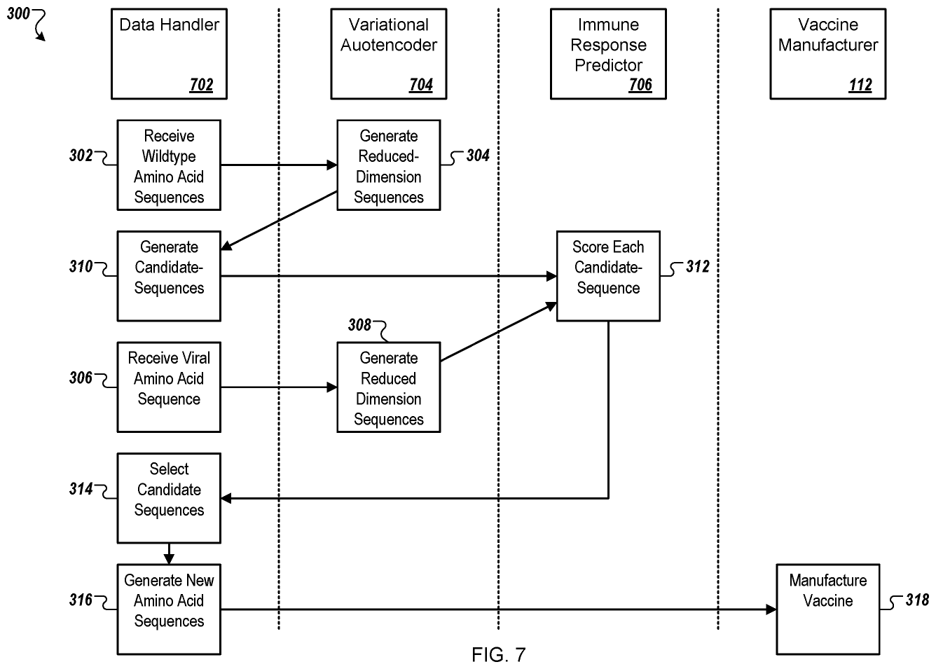

FIG. 7 is a swimlane diagram of the process 300 to manufacture a vaccine. To perform elements of the process 300, the computer system 106 can use operational elements such as a data handler 702, variational autoencoder 704, and immune response predictor 706, though other computational architecture can be used. Each element 702-706 may be embodied as one or more programs, routines, libraries, modules, etc. that execute in the computer system 106 and are able to communicate, store, and operate on data such as the data shown in FIGS. 1 and 2. As will be appreciated, the various elements 702-706 may be run on hardware that is remote from hardware running other elements of the computing system 106.

The data handler 702 operates in the computing system 106 to handle data operations such as accessing data on disk, transferring data over network connections within the computing system 106, and to manipulate data such as in the 302, 310, 314, and 316 in addition to other operations.

The variational autoencoder 704 includes one or more computational models such as an linear support vector machine (linear SVM), boosting for other algorithms (e.g., AdaBoost), neural networks, logistic regression, naïve Bayes, memory-based learning, random forests, bagged trees, decision trees, boosted trees, or boosted stumps. These models can operate on input data of a given dimensionality and produce output data respective of the input data in a lower dimensionality. The variational autoencoder 704 can compress the input information into a constrained multivariate latent distribution, and can also reconstruct such data back into the format of the input. Some implementations of the variational autoencoder can operate on input data characterized by an unknown probability distribution and approximate that data's distribution. Interstitial operations of the encoding and reconstructing functionality include, but are not limited to, predicting the mean and variance values for input data.

The titer predictor 706 includes one or more computational models such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps. These models may have been trained on data sets of sequences in the low-dimensions space that have been tagged with a result value indicating biological response such as an antibody titer when the sequence has been introduced to a subject (e.g., a human, a mammal, a patient).

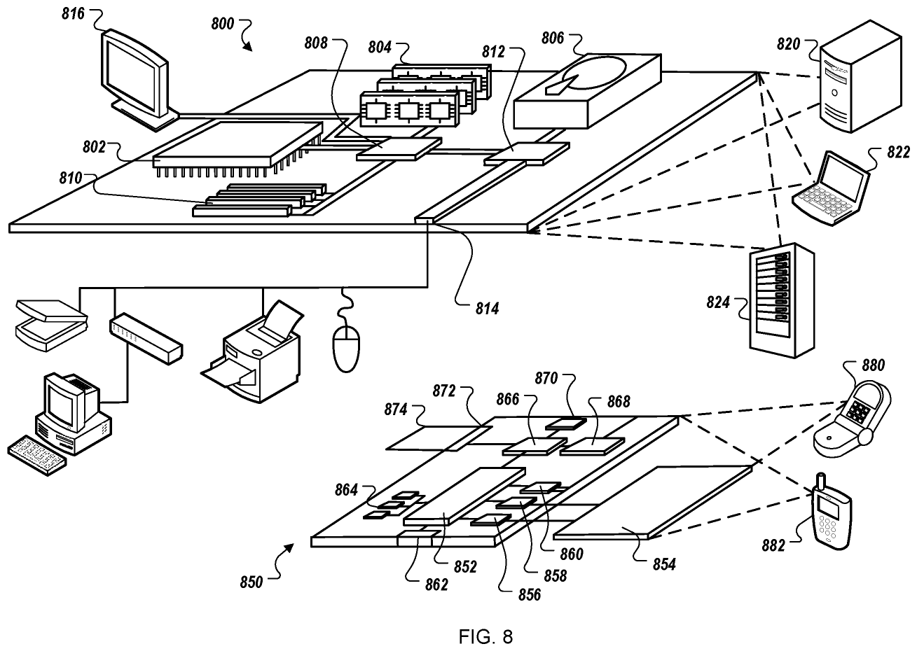

FIG. 8 shows an example of a computing device 800 and an example of a mobile computing device that can be used to implement the techniques described here. The computing device 800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and can be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 can also be another form of computer-readable medium, such as a magnetic or optical disk.

The storage device 806 is capable of providing mass storage for the computing device 800. In some implementations, the storage device 806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 804, the storage device 806, or memory on the processor 802.

The high-speed interface 808 manages bandwidth-intensive operations for the computing device 800, while the low-speed interface 812 manages lower bandwidth-intensive operations. Such allocation of functions is exemplary only. In some implementations, the high-speed interface 808 is coupled to the memory 804, the display 816 (e.g., through a graphics processor or accelerator), and to the high-speed expansion ports 810, which can accept various expansion cards (not shown). In the implementation, the low-speed interface 812 is coupled to the storage device 806 and the low-speed expansion port 814. The low-speed expansion port 814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 800 can be implemented in a number of different forms, as shown in the figure. For example, it can be implemented as a standard server 820, or multiple times in a group of such servers. In addition, it can be implemented in a personal computer such as a laptop computer 822. It can also be implemented as part of a rack server system 824. Alternatively, components from the computing device 800 can be combined with other components in a mobile device (not shown), such as a mobile computing device 850. Each of such devices can contain one or more of the computing device 800 and the mobile computing device 850, and an entire system can be made up of multiple computing devices communicating with each other.

The mobile computing device 850 includes a processor 852, a memory 864, an input/output device such as a display 854, a communication interface 866, and a transceiver 868, among other components. The mobile computing device 850 can also be provided with a storage device, such as a micro-drive or other device, to provide additional storage. Each of the processor 852, the memory 864, the display 854, the communication interface 866, and the transceiver 868, are interconnected using various buses, and several of the components can be mounted on a common motherboard or in other manners as appropriate.

The processor 852 can execute instructions within the mobile computing device 850, including instructions stored in the memory 864. The processor 852 can be implemented as a chipset of chips that include separate and multiple analog and digital processors. The processor 852 can provide, for example, for coordination of the other components of the mobile computing device 850, such as control of user interfaces, applications run by the mobile computing device 850, and wireless communication by the mobile computing device 850.

The processor 852 can communicate with a user through a control interface 858 and a display interface 856 coupled to the display 854. The display 854 can be, for example, a TFT (Thin-Film-Transistor Liquid Crystal Display) display or an OLED (Organic Light Emitting Diode) display, or other appropriate display technology. The display interface 856 can comprise appropriate circuitry for driving the display 854 to present graphical and other information to a user. The control interface 858 can receive commands from a user and convert them for submission to the processor 852. In addition, an external interface 862 can provide communication with the processor 852, so as to enable near area communication of the mobile computing device 850 with other devices. The external interface 862 can provide, for example, for wired communication in some implementations, or for wireless communication in other implementations, and multiple interfaces can also be used.

The memory 864 stores information within the mobile computing device 850. The memory 864 can be implemented as one or more of a computer-readable medium or media, a volatile memory unit or units, or a non-volatile memory unit or units. An expansion memory 874 can also be provided and connected to the mobile computing device 850 through an expansion interface 872, which can include, for example, a SIMM (Single In Line Memory Module) card interface. The expansion memory 874 can provide extra storage space for the mobile computing device 850, or can also store applications or other information for the mobile computing device 850. Specifically, the expansion memory 874 can include instructions to carry out or supplement the processes described above, and can include secure information also. Thus, for example, the expansion memory 874 can be provide as a security module for the mobile computing device 850, and can be programmed with instructions that permit secure use of the mobile computing device 850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 864, the expansion memory 874, or memory on the processor 852. In some implementations, the computer program product can be received in a propagated signal, for example, over the transceiver 868 or the external interface 862.

The mobile computing device 850 can communicate wirelessly through the communication interface 866, which can include digital signal processing circuitry where necessary. The communication interface 866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 868 using a radio-frequency. In addition, short-range communication can occur, such as using a Bluetooth, WiFi, or other such transceiver (not shown). In addition, a GPS (Global Positioning System) receiver module 870 can provide additional navigation- and location-related wireless data to the mobile computing device 850, which can be used as appropriate by applications running on the mobile computing device 850.

The mobile computing device 850 can also communicate audibly using an audio codec 860, which can receive spoken information from a user and convert it to usable digital information. The audio codec 860 can likewise generate audible sound for a user, such as through a speaker, e.g., in a handset of the mobile computing device 850. Such sound can include sound from voice telephone calls, can include recorded sound (e.g., voice messages, music files, etc.) and can also include sound generated by applications operating on the mobile computing device 850.

The mobile computing device 850 can be implemented in a number of different forms, as shown in the figure. For example, it can be implemented as a cellular telephone 880. It can also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

1. A dimension-reducing method for generating amino acid sequences, the method being performed by a system of one or more computers and comprising:
receiving one or more data objects defining a plurality of wild-type amino acid sequences;
generating, from the one or more data objects, a plurality of reduced-dimension sequences in a reduced-dimension space, wherein:
each reduced-dimension sequence contains data respective of at least one of the wild-type amino acid sequences,
the reduced-dimension space is of a lower dimensionality than the wild-type amino acid sequences, and
the plurality of reduced-dimension sequences define a distribution of values along each dimension of the reduced-dimension space,
generating a plurality of candidate sequences in the reduced-dimension space using the plurality of reduced-dimension sequences;
receiving one or more data objects defining a viral amino acid sequence;
generating at least one reduced-dimension viral sequences in the reduced-dimension space;
providing, as input to a titer-predictor, each of the candidate sequences and at least one of the reduced-dimension viral sequences;
receiving, as output from the titer-predictor, a candidate-score for each of the candidate sequences;
selecting at least one candidate sequence from among the candidate sequences;
generating at least one new amino acid sequence for each of the selected candidate sequences; and
providing the generated at least one amino acid sequence;
wherein each of the generated amino acid sequences is suitable for manufacturing a respective vaccine comprising at least one of the group consisting of i) a protein defined by the generated amino acid sequence, ii) a nucleic acid capable of producing the protein defined by the generated amino acid sequence, and iii) a delivery vehicle capable of producing the protein defined by the generated amino acid sequence.

2. The method of claim 1, wherein generating a plurality of reduced-dimension sequences comprises creation of representations of the wild-type amino acid sequences using a variational autoencoder that predicts mean and variance values of input data.

3. The method of claim 1, wherein each of the reduced-dimension sequences includes a respective group of values, and generating the plurality of candidate sequences in the reduced-dimension space comprises sampling distributions of values of the plurality of reduced-dimension sequences.

4. The method of claim 1, wherein the titer-predictor is configured to:
receive, as input, i) a first sequence in the reduced-dimension space and ii) a second sequence in the reduced-dimension space; and
provide, as output, a titer-score as the candidate score, the titer-score defines a measure of biological response between the first sequence and the second sequence.

5. The method of claim 1, wherein selecting the at least one candidate sequence as a selected candidate sequence comprises selecting N candidate sequences with the highest candidate-scores.

6. The method of claim 5, where Nis a value of 1, such that a single candidate sequence is selected.

7. The method of claim 5, where Nis a value greater than 1, such that a plurality of candidate sequences are selected.

8. The method of claim 1, wherein selecting the at least one candidate sequence as a selected candidate sequence comprises selecting candidate sequences with respective candidate-scores greater than a threshold value.

9. The method of claim 1, wherein each of the generated amino acid sequences is different from any of the wild-type amino acid sequences.

10. The method of claim 1, wherein at least one of the candidate sequences is in the plurality of reduced-dimension sequences.

11. The method of claim 1, wherein the respective vaccine is for one of the group consisting of i) influenza, ii) human rhinovirus, iii) HIV and iv) a coronavirus disease.

12. A system for generating amino acid sequences, the system comprising;
one or more processors; and
computer-memory storing instructions that, when executed by the processors, cause the processors to perform operations comprising:
receiving one or more data objects defining a plurality of wild-type amino acid sequences;
generating, from the one or more data objects, a plurality of reduced-dimension sequences in a reduced-dimension space, wherein:
each reduced-dimension sequence contains data respective of at least one of the wild-type amino acid sequences,
the reduced-dimension space is of a lower dimensionality than the wild-type amino acid sequences, and
the plurality of reduced-dimension sequences define a distribution of values along each dimension of the reduced-dimension space,
generating a plurality of candidate sequences in the reduced-dimension space using the plurality of reduced-dimension sequences;
receiving one or more data objects defining a viral amino acid sequence;
generating at least one reduced-dimension viral sequences in the reduced-dimension space;
providing, as input to a titer-predictor, each of the candidate sequences and at least one of the reduced-dimension viral sequences;
receiving, as output from the titer-predictor, a candidate-score for each of the candidate sequences;
selecting at least one candidate sequence from among the candidate sequences;
generating at least one new amino acid sequence for each of the selected candidate sequences; and
providing the generated at least one amino acid sequence,
wherein each of the generated amino acid sequences is suitable for manufacturing a respective vaccine comprising at least one of the group consisting of i) a protein defined by the generated amino acid sequence, ii) a nucleic acid capable of producing the protein defined by the generated amino acid sequence, and iii) a delivery vehicle capable of producing the protein defined by the generated amino acid sequence.

13. The system of claim 12, wherein generating a plurality of reduced-dimension sequences comprises creation of representations of the wild-type amino acid sequences using a variational autoencoder that predicts mean and variance values of input data.

14. The system of claim 12, wherein each of the reduced-dimension sequences includes a respective group of values, and generating the plurality of candidate sequences in the reduced-dimension space comprises sampling distributions of values of the plurality of reduced-dimension sequences.

15. The system of claim 12, wherein the titer-predictor is configured to:
receive, as input, i) a first sequence in the reduced-dimension space and ii) a second sequence in the reduced-dimension space; and
provide, as output, a titer-score as the candidate score, the titer-score defines a measure of biological response between the first sequence and the second sequence.

16. The system of claim 12, wherein selecting the at least one candidate sequence as a selected candidate sequence comprises selecting N candidate sequences with the highest candidate-scores.

17. A non-transitory, computer readable media storing instructions that, when executed by one or more processors, cause the one or more processors to perform operations comprising:
receiving one or more data objects defining a plurality of wild-type amino acid sequences;
generating, from the one or more data objects, a plurality of reduced-dimension sequences in a reduced-dimension space, wherein:
each reduced-dimension sequence contains data respective of at least one of the wild-type amino acid sequences,
the reduced-dimension space is of a lower dimensionality than the wild-type amino acid sequences, and
the plurality of reduced-dimension sequences define a distribution of values along each dimension of the reduced-dimension space,
generating a plurality of candidate sequences in the reduced-dimension space using the plurality of reduced-dimension sequences;
receiving one or more data objects defining a viral amino acid sequence;
generating at least one reduced-dimension viral sequences in the reduced-dimension space;
providing, as input to a titer-predictor, each of the candidate sequences and at least one of the reduced-dimension viral sequences;
receiving, as output from the titer-predictor, a candidate-score for each of the candidate sequences;
selecting at least one candidate sequence from among the candidate sequences;
generating at least one new amino acid sequence for each of the selected candidate sequences; and
providing the generated at least one amino acid sequence,
wherein each of the generated amino acid sequences is suitable for manufacturing a respective vaccine comprising at least one of the group consisting of i) a protein defined by the generated amino acid sequence, ii) a nucleic acid capable of producing the protein defined by the generated amino acid sequence, and iii) a delivery vehicle capable of producing the protein defined by the generated amino acid sequence.

18. The media of claim 17, wherein generating a plurality of reduced-dimension sequences comprises creation of representations of the wild-type amino acid sequences using a variational autoencoder that predicts mean and variance values of input data.

19. The media of claim 17, wherein each of the reduced-dimension sequences includes a respective group of values, and generating the plurality of candidate sequences in the reduced-dimension space comprises sampling distributions of values of the plurality of reduced-dimension sequences.

20. The media of claim 17, wherein the titer-predictor is configured to:
receive, as input, i) a first sequence in the reduced-dimension space and ii) a second sequence in the reduced-dimension space; and
provide, as output, a titer-score as the candidate score, the titer-score defines a measure of biological response between the first sequence and the second sequence.


SEQUENCE LISTING (1 sequence)

SEQ ID NO: 1 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRKSSSSFFSILNWLTHLNYTYPALNVTMPNKEQFDKLYIWGVHHPGTDKDQISLYARSSGRITVSTKRSQQAVIPNIGSRPRIRDIPSRISIYWTIVKPGDILLINSTGNLIAPRGYFKIRSGKSSIMRSDEPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI